(19) United States
(12) Patent Application Publication
(10) Pub. No.: US 2008/0188875 A1
(43) Pub. Date: Aug. 7, 2008

(54) BLOODLESS AND PAINLESS SURGICAL METHOD

(76) Inventor: Sarkis Yeretsian, Laval (CA)

Correspondence Address: OGILVY RENAULT LLP, 1981 MCGILL COLLEGE AVENUE, SUITE 1600, MONTREAL, QC, H3A2Y3, omitted

(21) Appl. No.: 11/671,606

(22) Filed: Feb. 6, 2007

(51) Int. Cl. A61B 17/122

(52) U.S. Cl. 606/157

(57) ABSTRACT

A method of arresting blood vessels in soft tissue at a predetermined location relative to an excision location where a part of the soft tissue is to be removed, is described. The method comprises securing at least one clip to a retention means of at least one of a pair of fingers of forceps. The fingers of the forceps are then positioned on a respective side of the tissue at the predetermined location. The fingers are displaced towards one another to cause the at least one clip to penetrate the soft tissue and form a clamp whereby to clamp the blood vessels at the predetermined location. Thereafter an excision is made to remove the portion of the soft tissue and the fingers of the forceps are withdrawn with the clamp retained engaged by the clip. The clip, device and method achieve a substantially bloodless and painless surgical procedure. The clip is constructed from bioabsorbable polymeric material and may be impregnated with an analgesic, anesthetic, an antibiotic or an anti-inflammatory substance.

### TECHNICAL FIELD

The present invention generally relates to cicatrisation of bodily soft tissue and, more particularly, to clips for joining parts of soft tissue, a device for applying the clips to clamp the parts of tissue, and a method for using the device to achieve substantially bloodless and painless surgical procedure.

### BACKGROUND ART

Traditionally, in the surgical field, wounds to bodily soft tissue are closed by stitching. Although stitching has proven to be effective, in several situations, the technique can be cumbersome and lengthy. Other methods and techniques are contemplated to reduce surgery time in given types of surgery and to avoid other complications.

As shown at www.jnjgateway.com, skin stapling devices have been provided as an alternative to stitching. The skin stapling devices apply staples to interconnect parts of soft tissue for cicatrisation. For instance, the Proximate Plus MD skin stapler, as shown from the above-cited website, is a single patient use instrument sold ready for use. The skin stapler uses stainless steel staples, and is therefore for external applications, as the staples must be removed after sufficient cicatrisation of the tissues has occurred.

### SUMMARY OF INVENTION

It is a feature of the present invention to provide novel clips for joining parts of bodily soft tissue for cicatrisation and providing for a substantially bloodless and painless surgical procedure.

It is a further feature of the present invention to provide a device for applying the clips of the present invention.

It is a still further feature of the present invention to provide a method for using the device for applying clips of the present invention.

According to the above features, from a broad aspect, the present invention provides a method of arresting blood vessels in soft tissue at a predetermined location relative to an excision location where a part of the soft tissue is to be removed. The method comprises the steps of securing at least one clip to a retention means of at least one of a pair of fingers of forceps. The fingers are positioned on a respective side of the tissue at the predetermined location. The fingers are then displaced towards one another to cause the at least one clip to penetrate the soft tissue and form a clamp whereby to clamp the blood vessels at the predetermined location.

According to a further broad aspect of the present invention there is provided a clip device for joining portions of bodily soft tissue, the clip device comprising at least two treading portions adapted to be inserted through portions of bodily soft tissue to be joined; a coupling portion connected to the at least two treading portions to secure the threading portions to the portions of bodily soft tissue; and at least one of an anesthetic, analgesic and an antibiotic drug in at least one of the treading portions and the coupling portion and releasable for at least one of pain relief and prevention of infection of the soft tissue.

According to a still further broad aspect of the present invention, there is provided a device for applying a clip to join portions of bodily soft tissue, comprising a forceps having a pair of members each pivotally mounted to one another and each defining a handle portion and a finger potion, the handle portion being operable to displace the finger portions in a scissor action; and retention means on at least one of the finger portions, the retention means adapted to receive a clip for joining portions of bodily soft tissue; wherein a closing action of the forceps will cause the clip to be secured to the portions of bodily soft tissue.

### BRIEF DESCRIPTION OF DRAWINGS

A preferred embodiment of the present invention will now be described with reference to the accompanying drawings in which:

FIG. 1 is a perspective view of the device for applying a clip in accordance with a basic configuration of the present invention;

FIG. 2A is a perspective view of a first arm of the device;

FIG. 2B is a perspective view of a second arm of the device;

FIG. 3 is a perspective view of the device for applying a clip having sinuous clip engaging surfaces;

FIG. 4 is an enlarged perspective view of one of the sinuous clip engaging surfaces of the device of FIG. 3;

FIG. 5 is an enlarged perspective view of fingers of the device for applying clips having channeled clip engaging surfaces;

FIG. 6 is a schematic front elevation view of an outline of part of a sinuous clip portion constructed in accordance with the present invention, for use with the device of FIG. 3;

FIG. 7 is a front elevation view of a staple clip constructed in accordance with the present invention, to be used with the device of FIG. 5;

FIG. 8 is a front elevation view of a bank of staple clips constructed in accordance with the present invention, to be used with the device of FIG. 5;

FIG. 9 is a front elevation view of the staple clip of FIG. 7 having been bent by the device of FIG. 5;

FIG. 10 is a perspective view of another clip constructed in accordance with the present invention, to be used with the device of FIG. 5;

FIG. 11 is a front elevation view of the clip of FIG. 10;

FIG. 12 is a enlarged view of fingers of an embodiment of the device of FIG. 5;

FIG. 13 is a perspective view, from a downward point of view, of the device for applying a clip in accordance with a further embodiment of the present invention;

FIG. 14 is a perspective view, from a lateral point of view, of the device for applying a clip of FIG. 13;

FIG. 15 is an enlarged perspective view, from the downward point of view, of an operative portion of the device for applying a clip of FIG. 13, with a clip therein;

FIG. 16 is an enlarged perspective view, partly sectioned, from the downward point of view, of fingers of the operative portion of the device for applying a clip of FIG. 13, with a clip therein;

FIG. 17 is a perspective view of a clip constructed in accordance with a further embodiment of the present invention;

FIG. 18 is an exploded view of the clip of FIG. 17, with layers of soft tissue therebetween;

FIG. 19 is an enlarged perspective view of the clip of FIG. 17; and

FIG. 20 is an enlarged perspective view, from the lateral point of view, of the operative portion of the device for applying a clip of FIG. 13, with the clip of FIG. 17 therein.

### DESCRIPTION OF PREFERRED EMBODIMENTS

An alternative to stitching consists in providing a bio-absorbable clip that will resolve with time, along with a device for applying the clip. The clip and device combination can be used in given types of surgical procedures. For instance, the use of the clip and device combination is contemplated for the “Closed Bloodless Hemorrhoidectomy Method” as described in co-pending U.S. application Ser. No. 10/152,563, filed on May 23, 2002, by the present inventor.

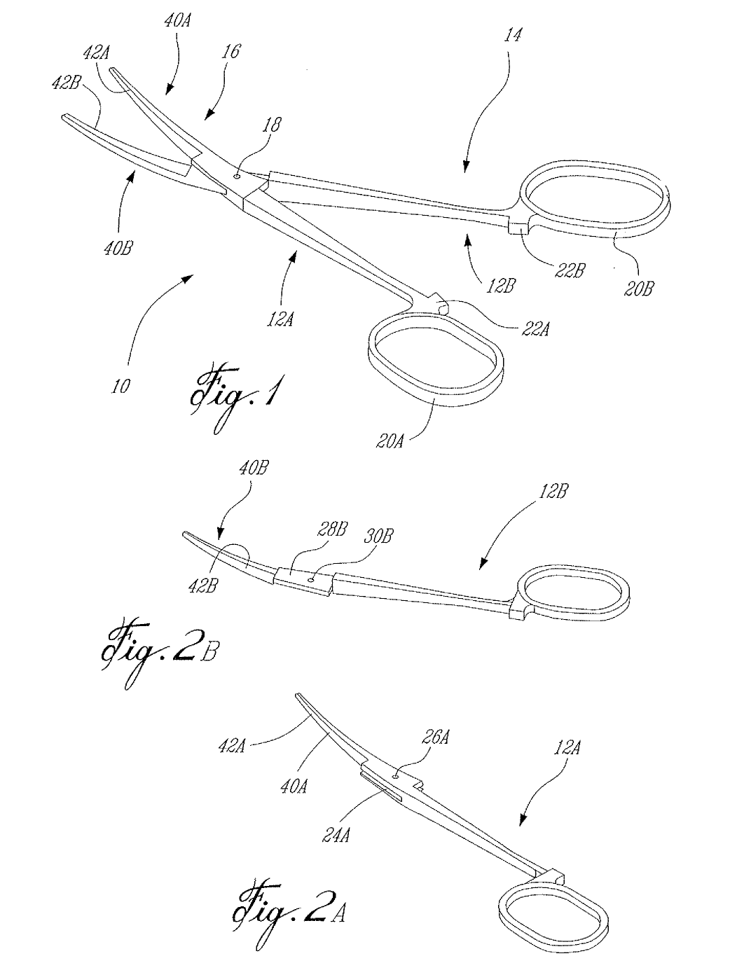

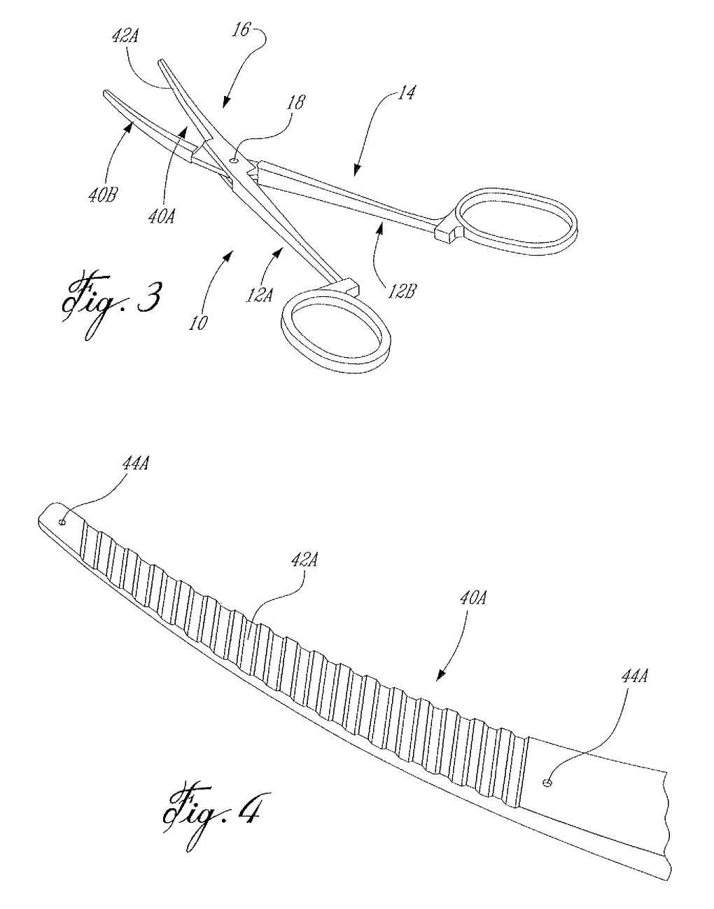

Referring to the drawings and, more particularly to FIGS. 1, 2A and 2B, a device for applying a clip constructed in accordance with a basic configuration of the present invention is generally shown at 10. The device 10 is made of a material (e.g., such as stainless steel, titanium) adapted for high-pressure autoclave, as it will be reusable and will thus require to be sterilizable. The device 10 has a forceps defined by a first arm 12A and a second arm 12B. For clarity purposes, components of the device 10 affixed with an “A” will be part of the first arm 12A, whereas components of the device 10 affixed with a “B” will be part of the second arm 12B.

The device 10 has a handle portion 14 and an operative portion 16. The first arm 12A and the second arm 12B are pivotally connected to one another at pivot 18 by a pivot pin, in a scissor-like fashion. The handle portion 14 and the operative portion 16 are separated by the pivot 18.

The handle portion 14 has finger engaging loops 20A and 20B in which fingers of a user person are inserted to handle/manipulate the operative end 16 of the device 10. Abutment stoppers 22A and 22B limit the motion of the device 10 in a closing direction thereof.

At the pivot 18, the first arm 12A has a slot 24A. The slot 24A is defined by a pair of parallel walls. A throughbore 26A is normal to the parallel walls and is provided for receiving the pivot 18. The second arm 12B has a connector wall portion 28B having a pair of parallel surfaces for sliding contact with the inner surface of the slot 24A when the arms 12A and 12B are coupled to one another. A throughbore 30B is normal to the parallel surfaces, and is coaxially positioned with respect to the throughbore 26A when the arms 12A and 12B are coupled to one another, so as to receive the pivot 18 therein.

The operative end 16 of the device 10 comprises fingers 40A and 40B. The fingers 40A and 40B have respective clip supporting surfaces 42A and 42B. The fingers 40A and 40B are curved with respect to a plane in which the device 10 generally lies. This feature is preferable as it increases the visibility of the fingers 40A and 40B by the surgeon. As also illustrated in FIGS. 1, 2A and 2B, the clip engaging surfaces 42A and 42B are substantially flat. However, as will be described hereinafter, the clip engaging surfaces 42A and 42B may have various patterns or shapes. The abutment stoppers 22A and 22B are positioned to come into contact one against the other generally simultaneously with the coplanar encounter of the clip engaging surfaces 42A and 42B.

Although not shown in FIGS. 1, 2A and 2B, the device 10 is provided with clip retention means on the clip engaging surfaces 42A and 42B, for securing clips to be applied during surgery. This will also be described in detail hereinafter during a description of given embodiments.

Referring to FIGS. 3 and 4, the device 10 is shown having a given pattern on the clip engaging surfaces 42A and 42B. More precisely, the patterns can be described as sinuous from a viewpoint normal to a plane in which both fingers 40 lie. The patterns are out of phase with respect to one another so as to intermesh when the device 10 is in its closed position.

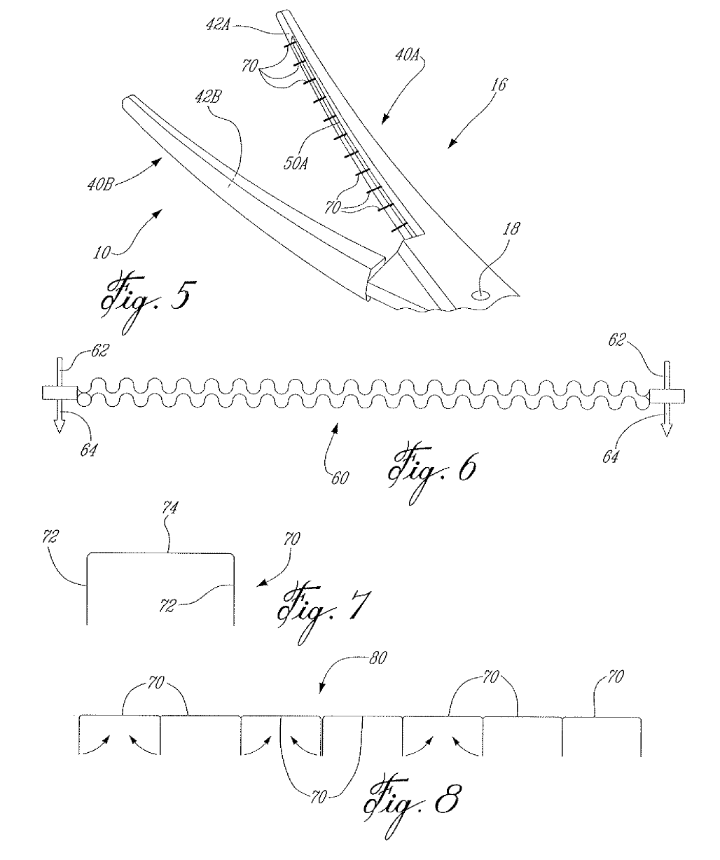

As seen in FIG. 4, a pair of connector holes 44A are provided at opposed ends of the pattern of the clip engaging surface 42A. The holes 44A are axially normal to the clip engaging surface 42A. Accordingly, a clip portion 60 having a pair of sinuous surfaces, as shown in FIG. 6, and provided with a pair of connector pins 62 at opposed ends thereof, can be releasably positioned against the clip engaging surface 42A with the connector pins 62 received in the connector holes 44A. A complementary clip portion (not shown) is retained on the clip engaging surface 42B in a similar fashion to the clip portion 60 (i.e., connector pins). Once the clip portion 60 and complementary clip portion are positioned on the device 10, the fingers 40A and 40B are closed against one another, whereby connection means (i.e., treading portion 64) on the clip portion 60 and complementary clip can cooperate to form a clip as a response to a closing action and pressure of the device 10, to secure soft tissue therebetween for cicatrisation. Once the clip portion 60 and the complementary clip are interconnected to squeeze soft tissue and surgical procedures are done (e.g., excision), the device 10 is opened, and the fingers 40A and 40B are thus separated from one another, thereby releasing the clip therefrom. The device 10 can thus receive another clip for further application.

Referring to FIG. 5, the device 10 has a channel 50A along its clip engaging surface 42A of the finger 40A. The channel 50A is adapted to receive and retain various types of clips.

As shown in FIG. 7, a staple clip, in accordance with the present invention, is generally shown at 70. The staple clip 70 has opposed treading portions 72 to puncture the soft tissue and a coupling portion 74 interconnecting the treading portions 72. The staple clip 70 is held in the channel 50A, by the coupling portion 74 being inserted in friction fit therein. In FIG. 5, a plurality of staples 70, in the form of a bank of staples as will be described hereinafter, is shown being inserted into the channel 50A. It is preferred that the channel 50A fully accommodates the staples 70 such that the surgeon is free to displace the device 10 on the soft tissue without the staples 70 interfering

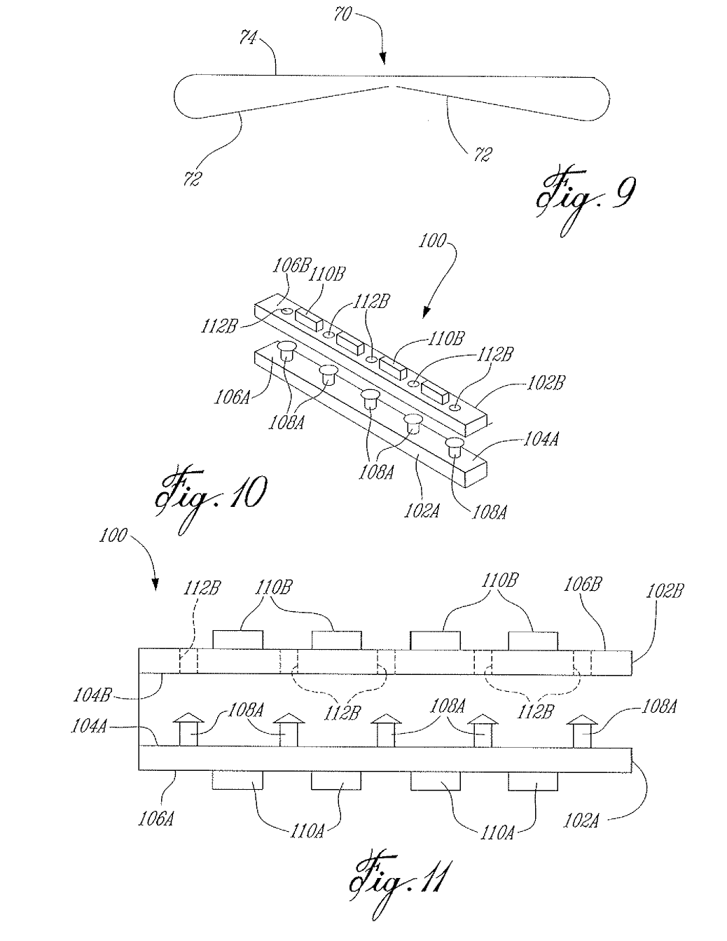

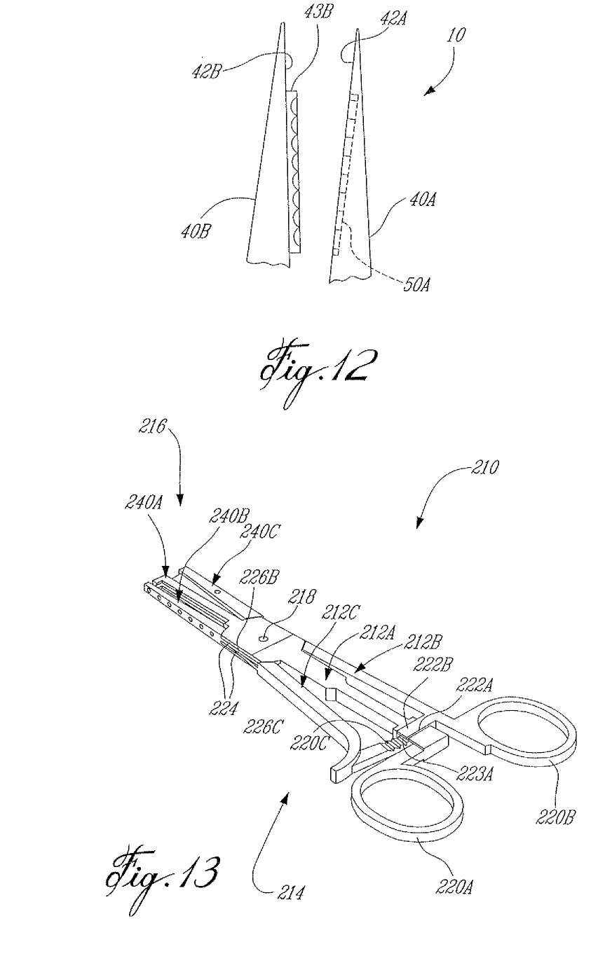

As seen in FIG. 12, the finger 40B has a protruding bar 43B with deflecting formations. Accordingly, when a closing action is performed on the device 10, the protruding bar 43B enters the cavity 50A with soft tissue being entrained therein. In doing so, the treading portions 72 of the staple clip 70 puncture the parts of soft tissue, to meet with the deflecting formations of the protruding bar 43B to cause the treading portions 72 to bend inwardly (similarly to a paper staple), as illustrated in FIG. 9 to provide securement and retention. Accordingly, soft tissue is squeezed between the bent treading portions 72 and the coupling portion 74, so as to facilitate cicatrisation.

As shown in FIG. 8, a bank of staple clips 70 is generally shown at 80, and consists of a plurality of side by side staple clips 70. The bank 80 can be used for joining soft tissue of a relatively straight wound.

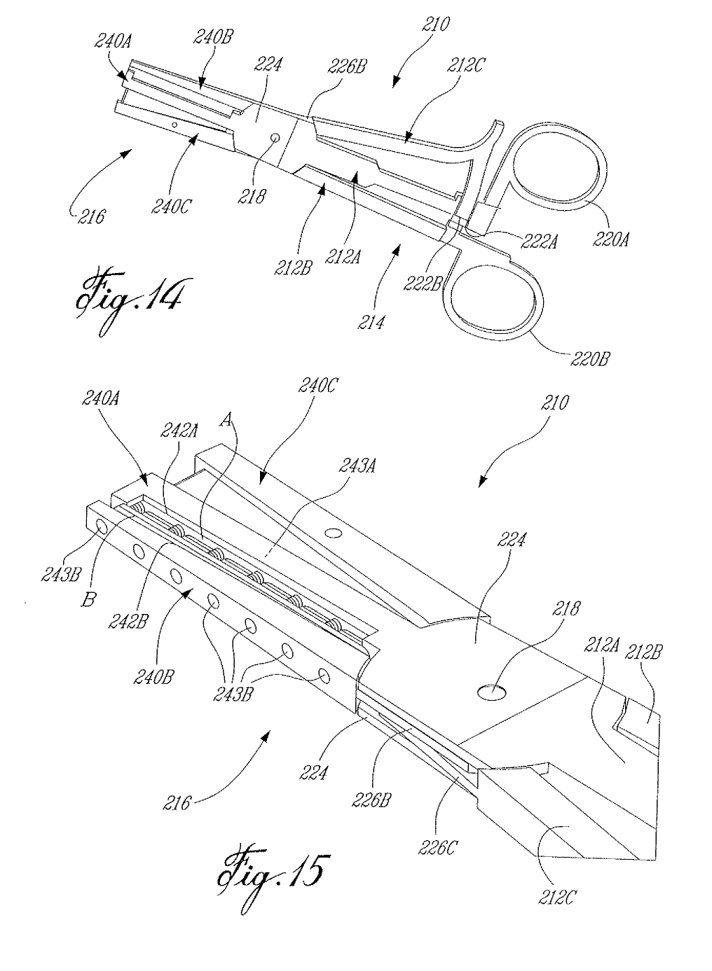

Referring to FIGS. 10 and 11, a clip adapted to be used with the device 10 of FIG. 5 is generally shown at 100. The clip 100 has a male portion 102A and a female portion 102B. The male portion 102A has a body having a contact surface 104A and an applying surface 106A. A plurality of treading portions 108A are normal to the contact surface 104A, whereas a plurality of connectors 110A are normal to the applying surface 106A. The treading portion 108A each consist of a stem having a pointed head at a free end thereof. The pointed head is required to puncture soft tissue.

The female portion 102B has a body having a contact surface 104B and an applying surface 106B. The female portion 102B is similar to the male portion 102A as it has a plurality of connectors 110B normal to the applying surface. The female portion 102B differs from the male portion 104B in that it is provided with throughbores 112B.

As shown in FIG. 10, the spacing between the throughbores 112B is equivalent to the spacing between the treading portions 108A, such that the male portion 102A and the female portion 102B can be interconnected by cooperation of the treading portions 108A and the throughbores 112B. The pointed heads of the treading portions 108A is preferably larger in diameter than the throughbores 112B to ensure that the connectors 102A and 102B remain connected to one another, with parts of soft tissue squeezed between the contact surfaces 104A and 104B for cicatrisation. The clip 100 preferably consists of a biodegradable polymer, whereby it has some level of resiliency, such that the pointed heads can squeeze through the throughbores 112B even though they are of larger diameter.

The clips 100 are applied to soft tissue by way of the connectors 110A and 110B being received in the channel 50A in FIG. 5 and a corresponding channel (not shown) of the finger 40B. The channels 50 prevent lateral displacement of the clips 100 with respect to the clip engaging surfaces 42 and provide perfect alignment of the male and female portions of the clip. A closing action of the device 10 causes the male portion 102A and the female portion 102B to interconnect with soft tissue squeezed between the contact surfaces 104A and 101B. Once the portion 102A and 102B are interconnected, the device 10 can be opened to release the portions 102A and 102B.

It is also contemplated to provide the device 10 with snap-fit locking means to releasably secure the clip portion thereto. The snap-fit locking means would be adapted to release the clip upon separation of the fingers 40A and 40B from one another. The interconnection force between the portions of the clip would be above the force required the snap-fit force retaining the clip portions to the device 10.

As described in U.S. application Ser. No. 10/152,563, some types of surgical procedures involve joining parts of soft tissue prior to proceeding with an excision. Accordingly, a use of the clips and device of the present invention is contemplated for closed bloodless excision surgery. In such a case, the device 10 can be kept in place to apply pressure on the clips to ensure proper hemostasis.

The clips described in FIGS. 6 to 11 are preferably made of a bio-absorbable polymeric material and are coated or may encapsulate a pharmaceutical agent, such as an anesthetic, an analgesic and/or an antibiotic for gradual local delivery to the soft tissue being held for cicatrisation. An anti-inflammatory agent may also be encapsulated. For instance, an antibiotic such as Cipro™ can be used for the treatment of acute hemorrhoidal disease. A suitable anesthetic is Xylocaine™, and suitable analgesics are Celebrex™ and Toradol™. In the case of encapsulated agents, the biodegradation of the clips will cause the release of the agent, thereby delivering the agent locally. This will enable to avoid in most cases having to prescribe narcotics to the patient following surgery. The use of the clips in accordance with the present invention will substantially reduce the pain caused by the surgery in the case where an analgesic is used.

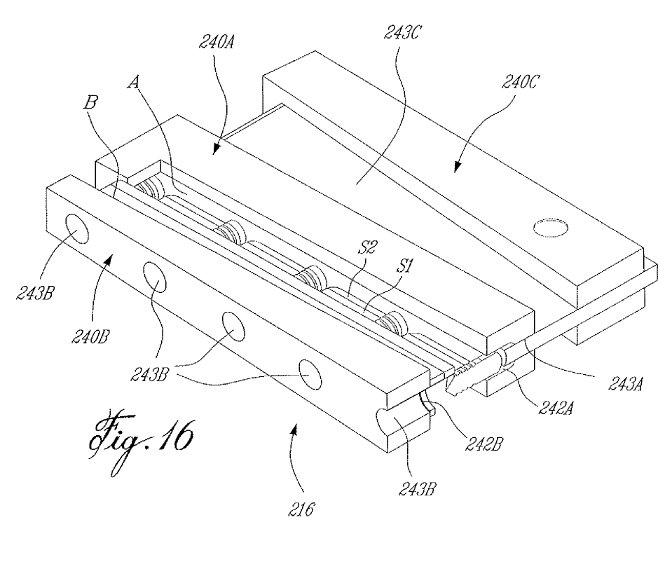

Referring to the drawings and, more particularly to FIGS. 13 and 14, a device for applying a clip constructed in accordance with another embodiment of the present invention is generally shown at 210. The device 210 is made of a material (e.g., such as stainless steel, titanium) adapted for high-pressure autoclave, as it will be reusable and will thus require to be sterilizable. The device 210 has a forceps defined by a first arm 212A, a second arm 212B, and a third arm 212C. For clarity purposes, components of the device 210 affixed with an “A” will be part of the first arm 212A, components of the device 210 affixed with a “B” will be part of the second arm 212B, and components of the device 210 affixed with a “C” will be part of the third arm 212C.

The device 210 has a handle portion 214 and an operative portion 216. The first arm 212A, the second arm 212B and the third arm 212C are pivotally connected to one another at pivot 218 by a pivot pin, in a scissor-like fashion. The first arm 212A and the second arm 212B will be used to position bodily soft tissue to be cicatrized between clip portions, as will be described hereinafter. The third arm 212C will be used to lock the clip portions to one another, with the bodily soft tissue to be cicatrized therebetween.

The handle portion 214 and the operative portion 216 are separated by the pivot 218. The first arm 212A holds the pivot 218 between a pair of spaced plates 224 thereof. The second arm 212B and the third arm 212C have plate portions 226B and 226C, respectively, by which they are in pivoting connection with the pivot 218.

### First Arm 212A and Second Arm 212B

The handle portion 214 has finger engaging loops 220A and 220B in which fingers of a user person are inserted to manipulate the operative portion 216 of the device 210. Abutment channel 222A and abutment 222B limit the motion of the device 210 in a closing direction thereof.

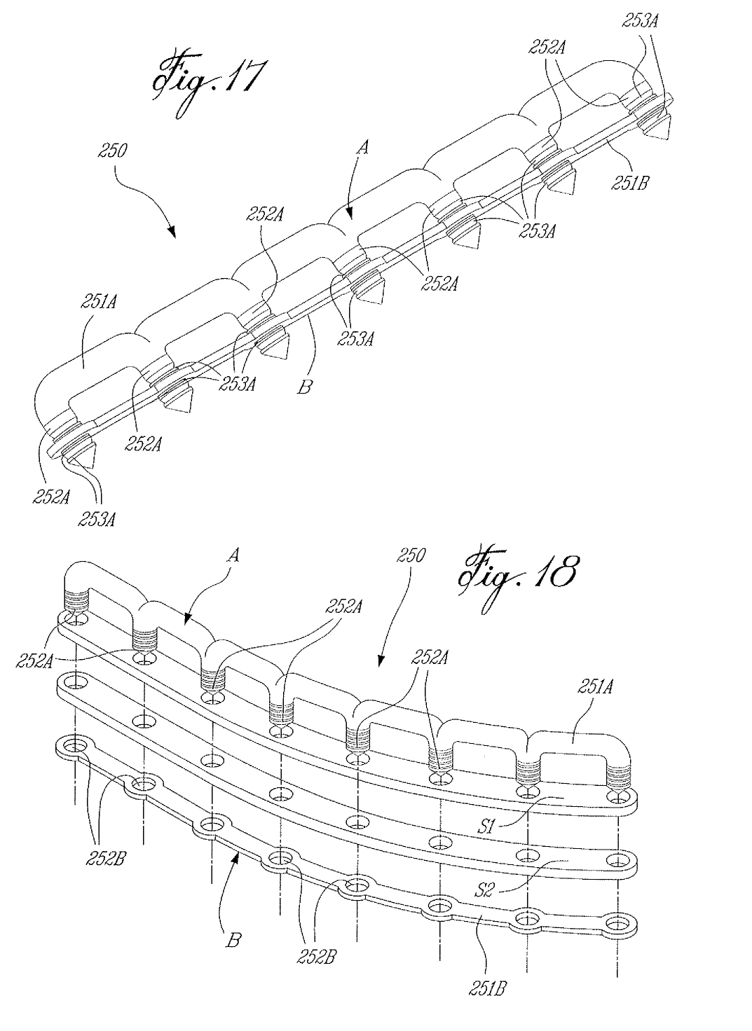

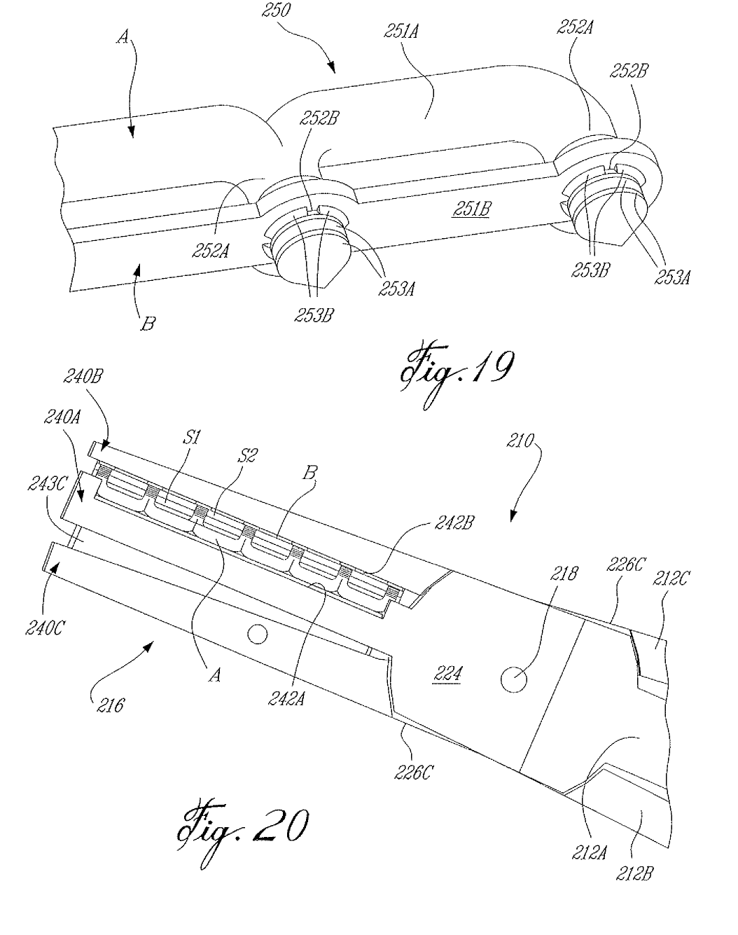

The operative portion 216 of the device 210 comprises fingers 240A, 240B and 240C. The fingers 240A and 240B have respective clip supporting portions 242A and 242B. More specifically, as shown in FIGS. 15 and 16, the clip supporting portion 242A is a receptacle in which a clip portion A is retained. As seen in FIG. 16, a slot 243A communicates with the clip supporting portion 242A, and will be used to remove the clip portion A from the clip supporting portion 242A, as will be described hereinafter.

The clip supporting portion 242B receives a clip portion B that is complementary to clip portion A, as will be described hereinafter. More specifically, the clip supporting portion 242B has a plurality of throughbores 243B.

### Third Arm 212C

Referring to FIG. 13, and as mentioned previously, the third arm 212C is used to lock the clip portions A and B together.

The third arm 212C has a ratchet portion 220C adjacent to the finger-engaging loops 220A and 220B. The ratchet portion 220C will cooperate with a retaining member 223A of the first arm 212A, such that the third arm 212C cannot be separated by scissor motion once the ratchet portion 220C and the retaining member 223A are engaged. A lateral pressure, normal to the plane of the device 210 is applied to separate the ratchet portion 220C from the retaining member 223A.

The third arm 212C has the finger 240C, which has a compressing member 243C. The compressing member 243C is sized so as to be received through the slot 243A, with a closing scissor motion between the first arm 212A and the third arm 212C.

### Clip 250

Referring to FIGS. 17 and 18, a clip in accordance with an embodiment of the present invention is generally shown at 250. The clip 250 has the clip portion A, and the clip portion B.

The clip portion A has a brace portion 251A, from which project male connectors 252A. The male connectors 252A each have a pointy end and a series of annular wedges 253A.

The clip portion B has a brace portion 251B, with a plurality of female connectors 252B. The female connectors 252B each have fins 253B that will cooperate with the annular wedges 253A to enable the reception and locking of the male connectors 252A in the female connectors 252B, as shown in FIG. 17, and enlarged in FIG. 19.

In FIG. 18, soft tissue layers to be cicatrized are shown schematically at S1 and S2. The clip 250 is similar to the clips described in FIGS. 6 to 11 in composition. Therefore, the clip 250 is preferably biodegradable so as to degrade over the cicatrization of the soft tissue layers S1 and 52.

Application of the Clip 250 with the Device 210

Referring concurrently to FIGS. 13, 16 and 18, clip portions A and B of the clip 250 are positioned in the clip supporting portions 242A and 242B, respectively. The fingers 240A and 240B are spaced from each other by the handle of the device 210, using the finger-engaging loops 220A and 220B. Layers of soft tissue to be cicatrized are positioned between the clips A and B, as illustrated in FIG. 18.

At this point, the fingers 240A and 240B can be brought together, once more by the leverage provided by the finger-engaging loops 220A and 220B. The layers of soft tissue are retained between the clip portions A and B. As mentioned previously, the motion of the fingers 240A and 240B is restricted by the abutment channel 222A and the abutment 222B (FIG. 13).

At this point, the clip portions A and B are locked to one another so as to retain the layers of soft tissue therebetween. This step is performed using the third arm 212C. The closing motion of the third arm 212C will result in the compressing member 243C entering the slot 243A, and pressing against the clip portion A. This pressure will result in the clip portions A and B interconnecting, with the soft tissue layers squeezed therebetween. The cooperation between the ratchet portion 220C and the retaining member 223A will keep the fingers 240A and 240C together.

Therefore, following the applied pressure of the third arm 212C, the clip 250 will be formed with the layers of soft tissue therebetween. The cooperation between the clip portions A and B will ensure that the clip 250 remains in position. The clip 250 will degrade over time, releasing an anesthetic or the like if required, and leaving the soft tissue cicatrized. It is pointed out that the clip and its application by the forceps of the invention results in the excision of tissue and which procedure is substantially bloodless and painless to the patient.

It is within the ambit of the present invention to cover any obvious modifications of the embodiments described herein, provided such modifications fall within the scope of the appended claims.

I claim:

1. A method of arresting blood vessels in soft tissue at a predetermined location relative to an excision location where a part of soft tissue is to be removed, said method comprising the steps of:
i) securing at least one clip to a retention means of at least one of a pair of fingers of forceps;
ii) positioning said fingers of said forceps on a respective side of said tissue at said predetermined location; and
iii) displacing said fingers towards one another to cause said at least one clip to penetrate said soft tissue and form a clamp whereby to clamp said blood vessels at said predetermined location.

2. A method as defined in claim 1 wherein said clip is a staple clip having opposed threading portions to puncture said soft tissue and a coupling interconnecting bridge portion, said step (i) comprising securing said bridge portion in a channel of said at least one of said fingers by friction fit therein, said step (iii) comprising causing said threading portions to deflect towards one another after penetrating said soft tissue to squeeze said tissue at said predetermined location to arrest said blood vessels and facilitate cicatrisation.

3. A method as defined in claim 2 wherein said step (i) comprises securing said bridge portion of at least two of said staple clips in aligned end-to-end relationship in said channel, the other of said pair of fingers having deflecting formations therein to cause said threading portions to deflect towards one another.

4. A method as defined in claim 1 wherein after said step (iii) there is provided the step (iv) of maintaining said clamp in clamping engagement with said soft tissue, (v) effecting an excision to remove said portion of said soft tissue, and (vi) displacing said fingers away from one another whereby said soft tissue is maintained clamped by said clip after said tissue portion has been removed in a substantially bloodless and painless surgical procedure.

5. A method as defined in claim 1 wherein said clip is constructed from bioabsorbable polymeric material, and wherein said material may be impregnated with one of an analgesic, an anesthetic or an antibiotic substance.

6. A method as defined in claim 1 wherein said clip is comprised of a male and female portion, said male portion having a tissue contacting surface and a plurality of threading portions normal to said contacting surface, said threading portions having a pointed free end, said female portion having a contacting surface and throughbores disposed to receive a connecting end of said threading portion therein, said step (i) comprising securing said male and female portions to a respective one of said fingers of said pair of fingers.

7. A method as defined in claim 4 wherein said forceps fingers are pivotally secured to one another and merge into a respective arm of a handle portion, said steps (ii) and (iii) being performed by operating said handle portion to displace said pair of fingers in a scissor action.

8. A method as defined in claim 7 wherein said forceps have a third arm provided with retention means cooperating with one of said fingers of said pair of fingers, said step (iv) comprising engaging said one of said fingers with said third arm by said retention means to maintain said clamping engagement of said forceps.

9. A clip device for joining portions of bodily soft tissue, said clip device comprising at least two threading portions adapted to be inserted through portions of bodily soft tissue to be joined to arrest blood vessels in said portion of bodily soft tissue, a coupling portion connected to the at least two threading portions to secure the threading portions to the portions of bodily soft tissue, and retention means to cause said threading portions to clamp said bodily soft tissue together to arrest said blood vessels.

10. A clip device as claimed in claim 9 wherein said retention means is constituted by a deflecting portion of said threading portions being bent inwardly towards one another and in a direction towards said coupling portion to squeeze said bodily soft tissue between said coupling portion and said bent deflecting portions, said clip being a staple clip.

11. A clip device as claimed in claim 10 wherein there is provided a plurality of said staple clips held together in a side-by-side relationship.

12. A clip device as claimed in claim 9 wherein said threading portions and coupling portion form part of a clip male portion, said retention means being constituted by a female clip portion, said coupling portion being a coupling body defining a contact surface and an applying surface, a plurality of said threading portions are disposed spaced apart on said contact surface and extend normal thereto, said female clip portion having a contact surface and an applying surface, and interconnecting means in said female clip portion to interconnect with said threading portion.

13. A clip device as claimed in claim 12 wherein said interconnecting means is constituted by a plurality of throughbores in said female clip portions for locking engagement with said threading portions with said contact surfaces facing one another.

14. A clip device as claimed in claim 10 wherein said clip is constructed of a biodegradable polymer.

15. A clip device as claimed in claim 12 wherein said clip is constructed of a biodegradable polymer.

16. A clip device as claimed in claim 14 wherein at least one of an anesthetic, analgesic and an antibiotic substance is integrated in at least one of the threading portions and the coupling portion and releasable for at least one of pain relief and prevention of infection of the soft tissue.

17. A clip device as claimed in claim 13 wherein said threading portions are straight stem members each having a pointed free end head to puncture said soft tissue, said free end head being a larger diameter than said throughbores.

18. A clip device as claimed in claim 12 wherein said applying surface of both said male and female clip portions are provided with two or more connector formations for positioning in a device to interconnect said clip portions together in perfect alignment.

19. A clip device as claimed in claim 17 wherein said straight stem members are provided with a series of annular wedges, said female clip portion being a flat strip member with each said throughbores having integrally formed fins to cooperate with said annular wedges to provide for adjustable securement means with associated ones of said straight stem members.

20. A device for applying a clip to clamp portions of bodily soft tissue together, said device comprising a forceps having a pair of arms each pivotally secured to one another and each defining a handle portion and a finger portion, the handle portion being operable to displace the finger portions in a scissor action, and retention means on at least one of the finger portions, the retention means adapted to receive a clip for joining portions of said bodily soft tissue, wherein a closing action of the forceps will cause the clip to be secured to clamp said portions of bodily soft tissue together.

21. A device as claimed in claim 20 wherein said device is constructed of sterilizable material.

22. A device as claimed in claim 20 wherein said handle portions are each provided with finger engaging means.

23. A device as claimed in claim 22 wherein said retention means is constituted by a clip retention slot formed in an engaging surface of said finger portion of a first of said pair of arms, the other of said finger portion having an engaging surface co-operating with said engaging surface of said first of said pair of arms to cause a clip retained in said clip retention slot to clampingly engage said portions of bodily soft tissue.

24. A device as claimed in claim 23 wherein said engaging surface of said other of said finger portion is provided with a deflection formation to cause a pair of threading portions of said clip to bend inwardly towards one another after they have penetrated said portion of bodily soft tissue.

25. A device as claimed in claim 23 wherein said finger portions are curved finger portions with respect to a common plane of said device and disposed in juxtaposition to one another, said curved finger portions providing increased visibility to said engaging surfaces.

26. A device as claimed in claim 24 wherein said deflecting formation is constituted by a sinus shaped surface to cause a plurality of said pair of threading portions to bend in a single closing action of said forceps.

27. A device as claimed in claim 22 wherein said pair of arms of said forceps constitutes a first and a second arm, there being provided a third arm, all said arms being pivotally connected to one another on a common pivot pin, said first and second arms engaging a respective clip portion and causing said clip portions to engage bodily soft tissue therebetween, said third arm locking said clip portions with one another in clamping engagement with said bodily soft tissue.

28. A device as claimed in claim 27 wherein said finger portions of said first and second arms are provided with respective clip supporting portions, said clip supporting portion of said finger portion of said first arm being a slot receptacle to retain a first clip portion having a plurality of threading portions.

29. A device as claimed in claim 28 wherein said clip supporting portion of said finger portion of said second arm is provided with a plurality of spaced-apart throughbores for receiving said plurality of threading portions of said first clip portion, a second clip portion supported on said clip supporting portion and having a plurality of holes aligned with said throughbores to receive an engaging end portion of said threading portions therethrough.

30. A device as claimed in claim 28 wherein said third arm has a ratchet portion intermediate said finger portion and said finger engaging means, said ratchet co-operating with a retaining member of said first arm whereby said third arm cannot be separated by said scissor motion when said ratchet portion and said retaining member are engaged.

31. A device as claimed in claim 30 wherein said third arm is provided with a compressing member sized for reception in said slot receptacle from a rear end thereof by a closing scissor motion between said first arm and said third arm.

32. A device as claimed in claim 30 wherein said ratchet member is separable from said retaining member by the application of a lateral pressure on said ratchet portion away from an engaging surface of said retaining member.
